(19) United States
(12) Patent Application Publication
(10) Pub. No.: US 2025/0225628 A1
(43) Pub. Date: Jul. 10, 2025

(54) DISTORTION CORRECTION OF LOW FIELD MAGNETIC RESONANCE IMAGES WITH PAIRED HIGH FIELD MAGNETIC RESONANCE IMAGES USING MACHINE LEARNING

(71) Applicant: NEURO42 INC., San Francisco, CA (US)

(72) Inventors: Benjamin INGLIS, San Francisco, CA (US); Carlo AMODEO, San Francisco, CA (US)

(73) Assignee: NEURO42 INC., San Francisco, CA (US)

(21) Appl. No.: 18/407,758

(22) Filed: Jan. 9, 2024

(51) Int. Cl. G06T 5/80; A61B 5/00; A61B 5/055; G06T 3/40; G06T 5/50

(52) U.S. Cl. CPC G06T 5/80; A61B 5/0042; A61B 5/055; A61B 5/7203; A61B 5/7267; G06T 3/40; G06T 5/50; G06T 2207/10088; G06T 2207/20081; G06T 2207/20084; G06T 2207/30004

(57) ABSTRACT

Disclosed is a system comprising a database storing a preoperative high-resolution image of an object of interest and a control circuit. The control circuit comprises a processor and a memory. The memory stores instructions executable by the processor to obtain a low-field strength magnetic resonance image of the object of interest. The memory stores further instructions executable by the processor to input the low-field strength MRI of the object of interest into a generator model of a pre-trained generative adversarial network. The generator model is pre-trained with low-field strength MRIs and paired high-resolution images to correct image distortions. The memory stores further instructions executable by the processor to output a distortion-corrected image of the object of interest from the generator model based on the low-field strength MRI and transmit the distortion-corrected image of the object of interest to a user interface.

### BACKGROUND

The present disclosure relates to magnetic resonance imaging (MRI), medical imaging, medical intervention, and surgical intervention. MRI systems often include large and complex machines that generate significantly high magnetic fields and create significant constraints on the feasibility of certain surgical interventions. Restrictions can include limited physical access to the patient by a surgeon and/or a surgical robot and/or limitations on the usage of certain electrical and mechanical components in the vicinity of the MRI scanner. Such limitations are inherent in the underlying design of many existing systems and are difficult to overcome.

### SUMMARY

In one general aspect, the present disclosure describes a system. The system comprising a database storing a preoperative high-resolution image of an object of interest and a control circuit comprising a processor and a memory. The memory stores instructions executable by the processor to obtain, intraoperatively, a low-field strength magnetic resonance image (MRI) of the object of interest. The memory stores further instructions executable by the processor to input, intraoperatively, the low-field strength MRI of the object of interest into a generator model of a pre-trained generative adversarial network, wherein the generator model is pre-trained with low-field strength MRIs and paired high-resolution images to correct image distortions. The memory stores further instructions executable by the processor to output, intraoperatively, a distortion-corrected image of the object of interest from the generator model based on the low-field strength MRI. The memory stores further instructions executable by the processor to transmit, intraoperatively, the distortion-corrected image of the object of interest to a user interface.

In at least one, the paired high-resolution images are images selected from a group consisting of a high-field strength MRI and a high-resolution computed tomography image.

In at least one aspect, the generative adversarial network is trained to a desired performance level using at least one set of training images, wherein each set of training images comprises a high-resolution training image of a training object of interest and low-field strength MRI training image of the training object of interest. In at least one aspect, the system further comprises a training control circuit to obtain, preoperatively, a first set of training images, wherein the first set of training images comprises a first low-field strength MRI training image and a first high-resolution training image of a first training object of interest. In at least one aspect, the training control circuit is further to input, preoperatively, the first low-field strength MRI training image into the generator model of the generative adversarial network to generate a first distortion-corrected training image. In at least one aspect, the training control circuit is further to input, preoperatively, the first distortion-corrected training image and the first high-resolution training image into a discriminator model of the generative adversarial network to evaluate the first training image. In at least one aspect, the training control circuit is further to update, preoperatively, one of the generator model and the discriminator model based on the evaluation of the first distortion-corrected training image by the discriminator model.

In at least one aspect, the discriminator model comprises a discriminator neural network and the generator model comprises a generator neural network, and wherein the training control circuit is further to adjust at least one weight of at least one layer of the discriminator neural network based on the discriminator model classifying the first distortion-corrected training image from the generator model as “real”.

In at least one aspect, the discriminator model comprises a discriminator neural network and the generator model comprises a generator neural network, and wherein the training control circuit is further to adjust at least one weight of at least one layer of the generator neural network of the generator model based on the discriminator model classifying the first distortion-corrected training image as “fake”.

In at least one aspect, the low-field strength MRI comprises a distortion of an anatomical structure depicted in the low-field strength MRI.

In at least one aspect, the paired high-resolution images comprise a first resolution, wherein the low-field strength MRIs comprises a second resolution, wherein the second resolution is less than the first resolution, wherein the memory stores further instructions executable by the processor to adjust the first resolution of the paired high-resolution images based on the second resolution of the low-field strength MRIs prior to training the generative adversarial network.

In at least one aspect, adjusting the first resolution based on the second resolution comprises smoothing each paired high-resolution image.

In at least one aspect, the memory stores further instructions executable by the processor to generate, intraoperatively, a low-field strength MRI with a low-field strength magnetic field.

In at least one aspect, the system further comprises a dome-shaped housing that is configured to house an array of magnets, wherein the array of magnets are arranged to generate the low-field strength magnetic field toward the object of interest within a field of view, wherein the low-field strength magnetic field comprises a magnetic field strength less than or equal to 1 T. In at least one aspect, the system further comprises a radio frequency coil assembly configured to selectively excite magnetization in the object of interest in the field of view.

In at least one aspect, the memory stores further instructions executable by the processor to transmit, intraoperatively, the distortion-corrected image of the object of interest to the user interface in real time.

In at least one aspect, the object of interest comprises an anatomical structure of a particular patient.

In another general aspect, the present disclosure describes a training system for a generative adversarial network. The training system comprising a training processor and a training memory storing a plurality of sets of training images, wherein each set of training images comprises a high-resolution training image of a training object of interest and a paired low-field strength MRI training image of the training object of interest. The memory stores instructions executable by the processor to obtain a first set of training images, wherein the first set of training images comprises a first low-field strength MRI training image and a first high-resolution training image of a first training object of interest, input the first low-field strength MRI training image into a generator model of a generative adversarial network to generate a first distortion-corrected training image, and input the first distortion-corrected training image and the first high-resolution training image into a discriminator model of the generative adversarial network. The memory stores instructions executable by the processor to evaluate, by the discriminator model, the first distortion-corrected training image to identify the first distortion-corrected training image as one of “real” or “fake”, and update, preoperatively, the generative adversarial network based on the evaluation of the first distortion-corrected training image by the discriminator model. Update the generative adversarial network comprises if the discriminator model classified the first distortion-corrected training image from the generator model as “real”, updating the discriminator model, and if the discriminator model classified the first distortion-corrected training image from the generator model as “fake”, updating the generator model.

In at least one aspect, the discriminator model comprises a discriminator neural network and the generator model comprises a generator neural network. In at least one aspect, the training memory stores instructions executable by the training processor to adjust at least one weight of at least one layer of the discriminator neural network of the discriminator model classified the first distortion-corrected training image from the generator model as “real”, and adjust at least one weight of at least one layer of the generator neural network of the generator model based on the discriminator model classifying the first distortion-corrected training image as “fake”.

In at least one aspect, the system further comprises training the generator model to a desired performance level by obtaining at least one other set of training images of a different subject and further training the generative adversarial network with the at least one other set of training images.

In another general aspect, the present disclosure describes a method comprises training, preoperatively, a generative adversarial network to a desired performance level. Training the generative adversarial network comprises inputting a first set of training images into the generative adversarial network. The first set of training images comprises a first low-field strength MRI training image and a first high-resolution training image of a first training object of interest, and wherein the generative adversarial network comprises a generator model and a discriminator model. Training the generative adversarial network further comprises generating, by the generator model, a first distortion-corrected training image based on the first low-field strength MRI training image, receiving, by the discriminator model, the first distortion-corrected training image and the first high-resolution training image, classifying, by the discriminator model, the first distortion-corrected training image as “real” or “fake”, and updating the generative adversarial network based on the classification. Updating the generative adversarial network comprises updating the discriminator model if the first distortion-corrected training image was classified as “real”, and updating the generator model if the first distortion-corrected training image was classified a “fake”. The method further comprising transmitting a notification to a user interface based on the generative adversarial network reaching the desired performance level.

In at least one aspect, the method further comprising obtaining, preoperatively, a high-field strength MRI of the first training object of interest.

In at least one aspect, the method further comprising obtaining, preoperatively, a high-resolution computed tomography image of the first training object of interest.

In at least one aspect, the method comprising, after training the generative adversarial network to the desired performance level, generating a distortion-corrected image with minimized distortions. Generating the distortion-corrected image comprises obtaining, intraoperatively, a low-field strength MRI of an object of interest with a low-field strength magnetic resonance imaging system. The low-field strength MRI comprises a dome-shaped housing and an array of magnets arranged about the dome-shaped housing. Generating the distortion-corrected image further comprises inputting, intraoperatively, the low-field strength MRI of the object of interest into the generator model, wherein the generator model is to generate the distortion-corrected image, and transmitting, intraoperatively, the distortion-corrected image to a user interface, wherein generation and transmission of the distortion-corrected image occurs in real-time.

In at least one aspect, the method further comprises projecting a low-field strength magnetic field from the array of magnets toward the object of interest located within a field of view, wherein the low-field strength magnetic field comprises a magnetic field strength less than or equal to 1 T, transmitting a radio frequency pulse sequence to a radio frequency coil assembly configured to selectively excite magnetization in the object of interest within the field of view, and receiving and recording an output signal from the radio frequency coil assembly.

### BRIEF DESCRIPTION OF THE DRAWINGS

The various aspects described herein, both as to organization and methods of operation, together with further objects and advantages thereof, may best be understood by reference to the following description, taken in conjunction with the accompanying drawings as follows.

FIG. 1 depicts components of a MRI scanning system including a dome-shaped housing for a magnetic array, the dome-shaped housing surrounding a region of interest therein and further depicting the dome-shaped housing positioned to receive at least a portion of the head of a patient reclined on the table into the region of interest, in accordance with at least one aspect of the present disclosure.

FIG. 2 is a perspective view of an alternative dome-shaped housing for a magnetic array for use with the MRI scanning system of FIG. 1, wherein access apertures are defined in the dome-shaped housing, in accordance with at least one aspect of the present disclosure.

FIG. 3 is a perspective view of an alternative dome-shaped housing for a magnetic array for use with the MRI scanning system of FIG. 1, wherein access apertures and an adjustable gap are defined in the dome-shaped housing, in accordance with at least one aspect of the present disclosure.

FIG. 4 depicts a dome-shaped housing for use with a MRI scanning system having an access aperture in the form of a centrally-defined hole, in accordance with at least one aspect of the present disclosure.

FIG. 5 is a cross-sectional view of the dome-shaped housing of FIG. 4, in accordance with at least one aspect of the present disclosure.

FIG. 6 depicts a control schematic for a MRI system, in accordance with at least one aspect of the present disclosure.

FIG. 7 is a method for obtaining imaging data from an MRI system, in accordance with at least one aspect of the present disclosure.

FIG. 8 depicts a MRI scanning system and a robotic system, in accordance with at least one aspect of the present disclosure.

FIG. 9 depicts a method for removing distortions from a LF-MRI using a paired image by applying a machine learning model, in accordance with at least one aspect of the present disclosure.

FIG. 10 depicts a method for training a generative adversarial network to remove distortions from a LF-MRI using a paired HF-MRI, in accordance with at least one aspect of the present disclosure.

FIG. 11 depicts a method for removing distortions from a LF-MRI during a surgical procedure, in accordance with at least one aspect of the present disclosure.

FIG. 12 is a block diagram of a system for removing distortions from a LF-MRI during a surgical procedure, in accordance with at least one aspect of the present disclosure.

Corresponding reference characters indicate corresponding parts throughout the several views. The exemplifications set out herein illustrate various disclosed embodiments, is one form, and such exemplifications are not to be construed as limiting the scope thereof in any manner.

### DETAILED DESCRIPTION

Applicant of the present application also owns the following patent applications, which are each herein incorporated by reference in their respective entireties:
International Patent Application No. PCT/US2022/72143, titled NEURAL INTERVENTIONAL MAGNETIC RESONANCE IMAGING APPARATUS, filed May 5, 2022;
U.S. patent application Ser. No. 18/057,207, titled SYSTEM AND METHOD FOR REMOVING ELECTROMAGNETIC INTERFERENCE FROM LOW-FIELD MAGNETIC RESONANCE IMAGES, filed Nov. 19, 2022;
U.S. patent application Ser. No. 18/147,418, titled MODULARIZED MULTI-PURPOSE MAGNETIC RESONANCE PHANTOM, filed Dec. 28, 2022;
U.S. patent application Ser. No. 18/147,542, titled INTRACRANIAL RADIO FREQUENCY COIL FOR INTRAOPERATIVE MAGNETIC RESONANCE IMAGING, filed Dec. 28, 2022;
U.S. patent application Ser. No. 18/147,556, titled DEEP LEARNING SUPER-RESOLUTION TRAINING FOR ULTRA LOW-FIELD MAGNETIC RESONANCE IMAGING, filed Dec. 28, 2022;
U.S. patent application Ser. No. 18/153,111, titled ACCELERATING MAGNETIC RESONANCE IMAGING USING PARALLEL IMAGING AND ITERATIVE IMAGE RECONSTRUCTION, filed Jan. 11, 2023;
U.S. patent application Ser. No. 18/153,175, titled FAST T2-WEIGHTED AND DIFFUSION-WEIGHTED CHIRPED-CPMG SEQUENCES, filed Jan. 11, 2023;
U.S. patent application Ser. No. 18/450,010, titled ITERATIVE SHIMMING FOR LOW-FIELD HEAD-OPTIMIZED MRI, filed Aug. 15, 2023; and
U.S. patent application Ser. No. 18/459,712, titled ACTIVE SHIMMING FOR LOW-FIELD MAGNETIC RESONANCE IMAGING, filed Sep. 1, 2023.

Before explaining various aspects of interventional magnetic resonance imaging devices in detail, it should be noted that the illustrative examples are not limited in application or use to the details of construction and arrangement of parts illustrated in the accompanying drawings and description. The illustrative examples may be implemented or incorporated in other aspects, variations and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects and/or examples.

Various aspects are directed to neural interventional magnetic resonance imaging (MRI) devices that allows for the integration of surgical intervention and guidance with an MRI. This includes granting physical access to the area around the patient as well as access to the patient's head with one or more access apertures. In addition, the neural interventional MRI device may allow for the usage of robotic guidance tools and/or traditional surgical implements. In various instances, a neural interventional MRI can be used intraoperatively to obtain scans of a patient's head and/or brain during a surgical intervention, such as a surgical procedure like a brain biopsy or neurosurgery.

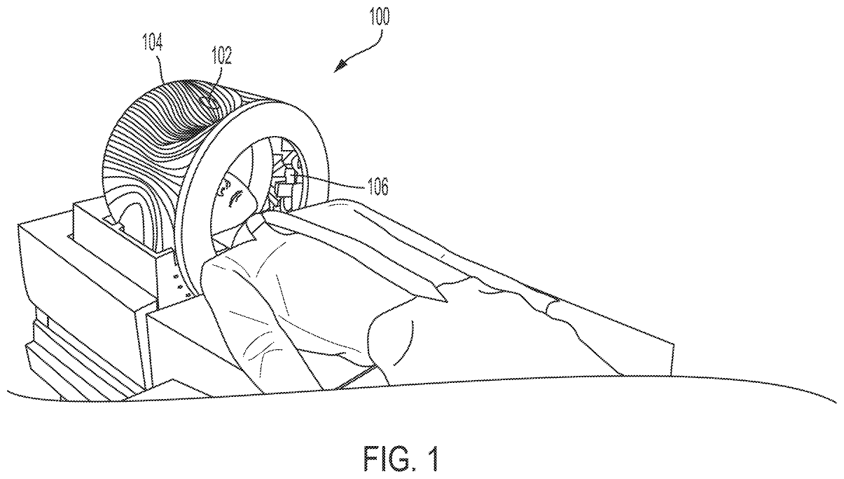

FIG. 1 depicts a MRI scanning system 100 that includes a dome-shaped housing 102 configured to receive a patient's head. The dome-shaped housing 102 can further include at least one access aperture configured to allow access to the patient's head to enable a neural intervention. A space within the dome-shaped housing 102 forms the region of interest for the MRI scanning system 100. Target tissue in the region of interest is subjected to magnetization fields/pulses, as further described herein, to obtain imaging data representative of the target tissue.

For example, a patient can be positioned such that his/her head is positioned within the region of interest within the dome-shaped housing 102. The brain can be positioned entirely within the dome-shaped housing 102. In such instances, to facilitate intracranial interventions (e.g. neurosurgery) in concert with MR imaging, the dome-shaped housing 102 can include one or more apertures that provide access to the brain. Apertures can be spaced apart around the perimeter of the dome-shaped housing.

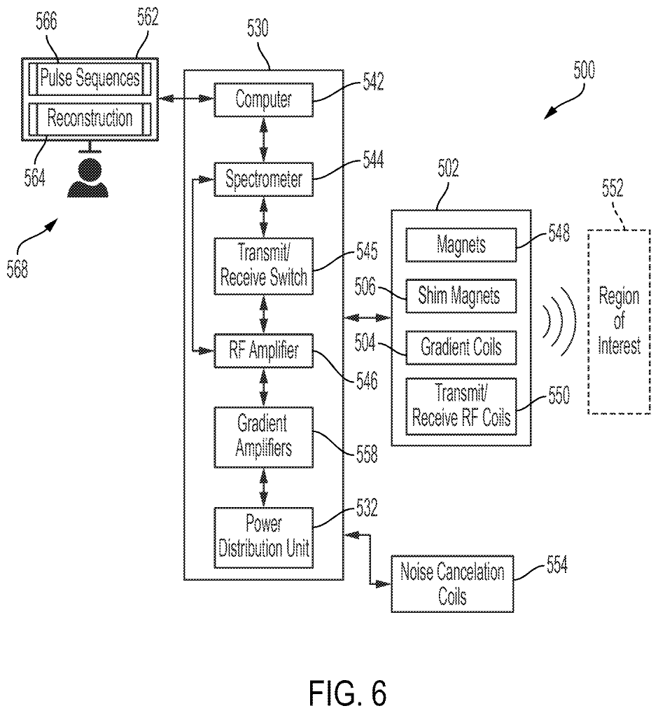

The MRI scanning system 100 can include an auxiliary cart (see, e.g. auxiliary cart 540 in FIG. 6) that houses certain conventional MRI electrical and electronic components, such as a computer, programmable logic controller, power distribution unit, and amplifiers, for example. The MRI scanning system 100 can also include a magnet cart that holds the dome-shaped housing 102, gradient coil(s), and/or a transmission coil, as further described herein. Additionally, the magnet cart can be attached to a receive coil in various instances. Referring primarily to FIG. 1, the dome-shaped housing 102 can further include RF transmission coils, gradient coils 104 (depicted on the exterior thereof), and shim magnets 106 (depicted on the interior thereof). Alternative configurations for the gradient coil(s) 104 and/or shim magnets 106 are also contemplated. In various instances, the shim magnets 106 can be adjustably positioned in a shim tray within the dome-shaped housing 102, which can allow a technician to granularly configure the magnetic flux density of the dome-shaped housing 102.

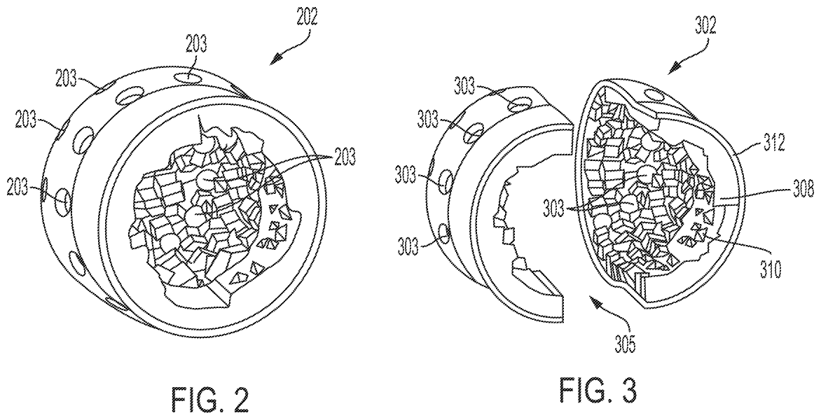

Various structural housings for receiving the patient's head and enabling neural interventions can be utilized with a MRI scanning system, such as the MRI scanning system 100. In one aspect, the MRI scanning system 100 may be outfitted with an alternative housing, such as a dome-shaped housing 202 (FIG. 2) or a two-part housing 302 (FIG. 3) configured to form a dome-shape. The dome-shaped housing 202 defines a plurality of access apertures 203; the two-part housing 302 also defines a plurality of access apertures 303 and further includes an adjustable gap 305 between the two parts of the housing.

In various instances, the housings 202 and 302 can include a bonding agent 308, such as an epoxy resin, for example, that holds a plurality of magnetic elements 310 in fixed positions. The plurality of magnetic elements 310 can be bonded to a structural housing 312, such as a plastic substrate, for example. In various aspects, the bonding agent 308 and structural housing 312 may be non-conductive or diamagnetic materials. Referring primarily to FIG. 3, the two-part housing 302 comprises two structural housings 312. In various aspect, a structural housing for receiving the patient's head can be formed from more than two sub-parts. The access apertures 303 in the structural housing 312 provide a passage directly to the patient's head and are not obstructed by the structural housing 312, bonding agent 308, or magnetic elements 310. The access apertures 303 can be positioned in an open space of the housing 302, for example.

There are many possible configurations of neural interventional MRI devices that can achieve improved access for surgical intervention. Many configurations build upon two main designs, commonly known as the Halbach cylinder and the Halbach dome described in the following article: Cooley et al. (e.g. Cooley, C. Z., Haskell, M. W., Cauley, S. F., Sappo, C., Lapierre, C. D., Ha, C. G., Stockmann, J. P., & Wald, L. L. (2018). Design of sparse Halbach magnet arrays for portable MRI using a genetic algorithm. IEEE transactions on magnetics, 54 (1), 5100112. The article “Design of sparse Halbach magnet arrays for portable MRI using a genetic algorithm” by Cooley et al., published in IEEE transactions on magnetics, 54 (1), 5100112 in 2018, is incorporated by reference herein in its entirety.

In various instances, a dome-shaped housing for an MRI scanning system, such as the system 100, for example, can include a Halbach dome defining a dome shape and configured based on several factors including main magnetic field B₀ strength, field size, field homogeneity, device size, device weight, and access to the patient for neural intervention. In various aspects, the Halbach dome comprises an exterior radius and interior radius at the base of the dome. The Halbach dome may comprise an elongated cylindrical portion that extends from the base of the dome. In one aspect, the elongated cylindrical portion comprises the same exterior radius and interior radius as the base of the dome and continues from the base of the dome at a predetermined length, at a constant radius. In another aspect, the elongated cylindrical portion comprises a different exterior radius and interior radius than the base of the dome (see e.g. FIGS. 2 and 3). In such instances, the different exterior radius and interior radius of the elongated cylindrical portion can merge with the base radii in a transitional region.

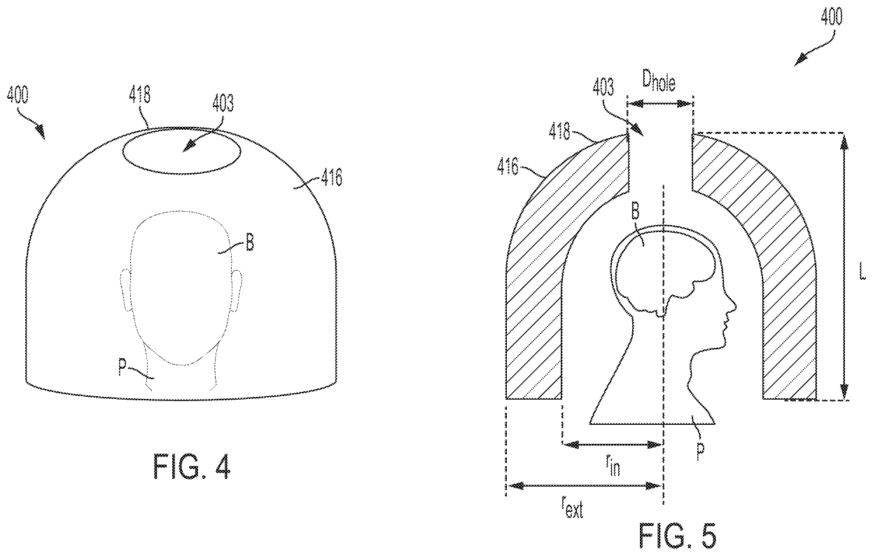

FIG. 4 illustrates an exemplary Halbach dome 400 for an MRI scanning system, such as the system 100, for example, which defines an access aperture in the form of a hole or access aperture 403, where the dome 400 is configured to receive a head and brain B of the patient P within the region of interest therein, and the access aperture 403 is configured to allow access to the patient P to enable neural intervention with a medical instrument and/or robotically-controlled surgical tool, in accordance with at least one aspect of the present disclosure. The Halbach dome 400 can be built with a single access aperture 403 at the top side 418 of the dome 400, which allows for access to the top of the skull while minimizing the impact to the magnetic field. Additionally or alternatively, the dome 300 can be configured with multiple access apertures around the structure 416 of the dome 400, as shown in FIGS. 2 and 3.

The diameter Dₕₒₗₑ of the access aperture 403 may be small (e.g. about 2.54 cm) or very large (substantially the exterior rₑₓₜ diameter of the dome 400). As the access aperture 403 becomes larger, the dome 400 begins to resemble a Halbach cylinder, for example. The access aperture 403 is not limited to being at the apex of the dome 400. The access aperture 403 can be placed anywhere on the surface or structure 416 of the dome 400. In various instances, the entire dome 400 can be rotated so that the access aperture 403 can be co-located with a desired physical location on the patient P.

FIG. 5 depicts relative dimensions of the Halbach dome 400, including a diameter Dₕₒₗₑ of the access aperture 403, a length L of the dome 400, and an exterior radius Text and an interior radius rᵢₙ of the dome 400. The Halbach dome 400 comprises a plurality of magnetic elements that are configured in a Halbach array and make up a magnetic assembly. The plurality of magnetic elements may be enclosed by the exterior radius text and interior radius rᵢₙ in the structure 416 or housing thereof. In one aspect, example dimensions may be defined as: rᵢₙ=19.3 cm; rₑₓₜ=23.6 cm; L=38.7 cm; and 2.54 cm≤D<19.3 cm.

Based on the above example dimensions, a Halbach dome 400 with an access aperture 403 may be configured with a magnetic flux density B₀ of around 72 mT, and an overall mass of around 35 kg. It will be appreciated that the dimensions may be selected based on particular applications to achieve a desired magnetic flux density B₀, total weight of the Halbach dome 400 and/or magnet cart, and geometry of the neural intervention access aperture 403.

In various aspects, the Halbach dome 400 may be configured to define multiple access apertures 403 placed around the structure 416 of the dome 400. These multiple access apertures 403 may be configured to allow for access to the patient's head and brain B using tools (e.g., surgical tools) and/or a surgical robot.

In various aspects, the access aperture 403 may be adjustable. The adjustable configuration may provide the ability for the access aperture 403 to be adjusted using either a motor, mechanical assist, or a hand powered system with a mechanical iris configuration, for example, to adjust the diameter Dₕₒₗₑ of the access aperture 403. This would allow for configuration of the dome without an access aperture 403, conducting an imaging scan, and then adjusting the configuration of the dome 400 and mechanical iris thereof to include the access aperture 403 and, thus, to enable a surgical intervention therethrough.

Halbach domes and magnetic arrays thereof for facilitating neural interventions are further described in International Patent Application No. PCT/US2022/72143, titled NEURAL INTERVENTIONAL MAGNETIC RESONANCE IMAGING APPARATUS, filed May 5, 2022, which is incorporated by reference herein in its entirety.

Referring now to FIG. 6, a schematic for an MRI system 500 is shown. The MRI scanning system 100 (FIG. 1) and the various dome-shaped housings and magnetic arrays therefor, which are further described herein, for example, can be incorporated into the MRI system 500, for example. The MRI system 500 includes a housing 502, which can be similar in many aspects to the dome-shaped housings 102 (FIG. 1), 202 (FIG. 2), and/or 302 (FIG. 3), for example. The housing 502 is dome-shaped and configured to form a region of interest, or field of view, 552 therein. For example, the housing 502 can be configured to receive a patient's head in various aspects of the present disclosure.

The housing 502 includes a magnet assembly 548 having a plurality of magnets arranged therein (e.g. a Halbach array of magnets). In various aspect, the main magnetic field B₀, generated by the magnetic assembly 548, extends into the field of view 552, which contains an object (e.g. the head of a patient) that is being imaged by the MRI system 500.

The MRI system 500 also includes RF transmit/receive coils 550. The RF transmit/receive coils 550 are combined into integrated transmission-reception (Tx/Rx) coils. In other instances, the RF transmission coil can be separate from the RF reception coil. For example, the RF transmission coil(s) can be incorporated into the housing 502 and the RF reception coil(s) can be positioned within the housing 502 to obtain imaging data.

The housing 502 also includes one or more gradient coils 504, which are configured to generate gradient fields to facilitate imaging of the object in the field of view 552 generated by the magnet assembly 548, e.g., enclosed by the dome-shaped housing and dome-shaped array of magnetic elements therein. Shim trays adapted to receive shim magnets 506 can also be incorporated into the housing 502.

During the imaging process, the main magnetic field B₀ extends into the field of view 552. The direction of the effective magnetic field (B₁) changes in response to the RF pulses and associated electromagnetic fields transmitted by the RF transmit/receive coils 550. For example, the RF transmit/receive coils 550 may be configured to selectively transmit RF signals or pulses to an object in the field of view 552, e.g. tissue of a patient's brain. These RF pulses may alter the effective magnetic field experienced by the spins in the sample tissue.

The housing 502 is in signal communication with an auxiliary cart 530, which is configured to provide power to the housing 502 and send/receive control signals to/from the housing 502. The auxiliary cart 530 includes a power distribution unit 532, a computer 542, a spectrometer 544, a transmit/receive switch 545, an RF amplifier 546, and gradient amplifiers 558. In various instances, the housing 502 can be in signal communication with multiple auxiliary carts and each cart can support one or more of the power distribution unit 532, the computer 542, the spectrometer 544, the transmit/receive switch 545, the RF amplifier 546, and/or the gradient amplifiers 558.

The computer 542 is in signal communication with a spectrometer 544 and is configured to send and receive signals between the computer 542 and the spectrometer 544. When the object in the field of view 552 is excited with RF pulses from the RF transmit/receive coils 550, the precession of the object results in an induced electric current, or MR current, which is detected by the RF transmit/receive coils 550 and sent to the RF preamplifier 556. The RF preamplifier 556 is configured to boost or amplify the excitation data signals and send them to the spectrometer 544. The spectrometer 544 is configured to send the excitation data to the computer 542 for storage, analysis, and image construction. The computer 542 is configured to combine multiple stored excitation data signals to create an image, for example. In various instances, the computer 542 is in signal communication with at least one database 562 that stores reconstruction algorithms 564 and/or pulse sequences 566. The computer 542 is configured to utilize the reconstruction algorithms to generate an MR image 568.

From the spectrometer 544, signals can also be relayed to the RF transmit/receive coils 550 in the housing 502 via an RF power amplifier 546 and the transmit/receive switch 545 positioned between the spectrometer 544 and the RF power amplifier 546. From the spectrometer 544, signals can also be relayed to the gradient coils 560 in the housing 502 via a gradient power amplifier 558. For example, the RF power amplifier 546 is configured to amplify the signal and send it to RF transmission coils 560, and the gradient power amplifier 558 is configured to amplify the gradient coil signal and send it to the gradient coils 560.

In various instances, the MRI system 500 can include noise cancellation coils 554. For example, the auxiliary cart 530 and/or computer 542 can be in signal communication with noise cancellation coils 554. In other instances, the noise cancellation coils 554 can be optional. For example, certain MRI systems disclosed herein may not include supplemental/auxiliary RF coils for detecting and canceling electromagnetic interference, i.e. noise.

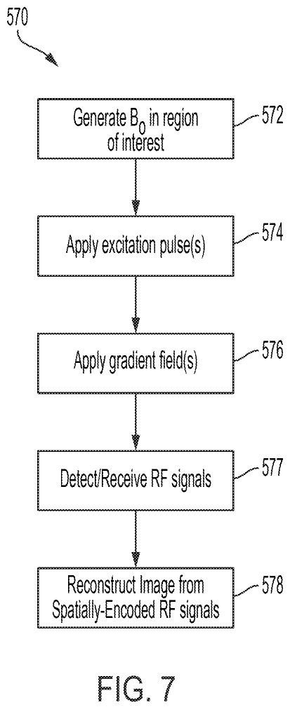

A flowchart depicting a process 570 for obtaining an MRI image is shown in FIG. 7. The flowchart can be implemented by the MRI system 500, for example. In various instances, at block 572, the target subject (e.g. a portion of a patient's anatomy), is positioned in a main magnetic field B₀ in a region of interest (e.g. region of interest 552), such as within the dome-shaped housing of the various MRI scanners further described herein (e.g. magnet assembly 548). The main magnetic field B₀ is configured to magnetically polarize the hydrogen protons (1H-protons) of the target subject (e.g. all organs and tissues) and is known as the net longitudinal magnetization Mo. It is proportional to the proton density (PD) of the tissue and develops exponentially in time with a time constant known as the longitudinal relaxation time T1 of the tissue. T1 values of individual tissues depend on a number of factors including their microscopic structure, on the water and/or lipid content, and the strength of the polarizing magnetic field, for example. For these reasons, the T1 value of a given tissue sample is dependent on age and state of health.

At block 574, a time varying oscillatory magnetic field B₁, i.e. an excitation pulse, is applied to the magnetically polarized target subject with a RF coil (e.g. RF transmit/receive coil 550). The carrier frequency of the pulsed B₁ field is set to the resonance frequency of the 1H-proton, which causes the longitudinal magnetization to flip away from its equilibrium longitudinal direction resulting in a rotated magnetization vector, which in general can have transverse as well as longitudinal magnetization components, depending on the flip angle used. Common B₁ pulses include an inversion pulse, or a 180-degree pulse, and a 90-degree pulse. A 180-degree pulse reverses the direction of the 1H-proton's magnetization in the longitudinal axis. A 90-degree pulse rotates the 1H-proton's magnetization by 90 degrees so that the magnetization is in the transverse plane. The MR signals are proportional to the transverse components of the magnetization and are time varying electrical currents that are detected with suitable RF coils. These MR signals decay exponentially in time with a time constant known as the transverse relaxation time T2, which is also dependent on the microscopic tissue structure, water/lipid content, and the strength of the magnetic field used, for example.

At block 576, the MR signals are spatially encoded by exposing the target subject to additional magnetic fields generated by gradient coils (e.g. gradient coils 560), which are known as the gradient fields. The gradient fields, which vary linearly in space, are applied for short periods of time in pulsed form and with spatial variations in each direction. The net result is the generation of a plurality of spatially encoded MR signals, which are detected at block 577, and which can be reconstructed to form MR images depicting slices of the examination subject. A RF reception coil (e.g. RF transmit/receive coil 550) can be configured to detect the spatially-encoded RF signals. Slices may be oriented in the transverse, sagittal, coronal, or any oblique plane.

At block 578, the spatially encoded signals of each slice of the scanned region are digitized and spatially decoded mathematically with a computer reconstruction program (e.g. by computer 542) in order to generate images depicting the internal anatomy of the examination subject. In various instances, the reconstruction program can utilize an (inverse) Fourier transform to back-transforms the spatially-encoded data (k-space data) into geometrically decoded data.

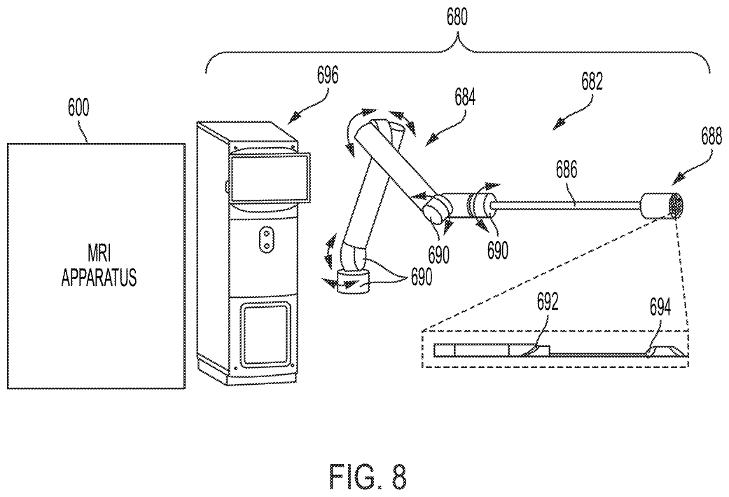

FIG. 8 depicts a graphical illustration of a robotic system 680 that may be used for neural intervention with an MRI scanning system 600. The robotic system 680 includes a computer system 696 and a surgical robot 682. The MRI scanning system 600 can be similar to the MRI system 500 and can include the dome-shaped housing and magnetic arrays having access apertures, as further described herein. For example, the MRI system 500 can include one or more access apertures defined in a Halbach array of magnets in the permanent magnet assembly to provide access to one or more anatomical parts of a patient being imaged during a medical procedure. In various instances, a robotic arm and/or tool of the surgical robot 682 is configured to extend through an access aperture in the permanent magnet assembly to reach a patient or target site. Each access aperture can provide access to the patient and/or surgical site. For example, in instances of multiple access apertures, the multiple access apertures can allow access from different directions and/or proximal locations.

In accordance with various embodiments, the robotic system 680 is configured to be placed outside the MRI system 600. As shown in FIG. 8, the robotic system 680 can include a robotic arm 684 that is configured for movements with one or more degrees of freedom. In accordance with various embodiments, the robotic arm 684 includes one or more mechanical arm portions, including a hollow shaft 686 and an end effector 688. The hollow shaft 686 and end effector 688 are configured to be moved, rotated, and/or swiveled through various ranges of motion via one or more motion controllers 690. The double-headed curved arrows in FIG. 8 signify exemplary rotational motions produced by the motion controllers 690 at the various joints in the robotic arm 684.

In accordance with various embodiments, the robotic arm 684 of the robotic system 682 is configured for accessing various anatomical parts of interest through or around the MRI scanning system 600. In accordance with various embodiments, the access aperture is designed to account for the size of the robotic arm 684. For example, the access aperture defines a circumference that is configured to accommodate the robotic arm 684, the hollow shaft 686, and the end effector 688 therethrough. In various instances, the robotic arm 684 is configured for accessing various anatomical parts of the patient from around a side of the magnetic imaging apparatus 600. The hollow shaft 686 and/or end effector 688 can be adapted to receive a robotic tool 692, such as a biopsy needle having a cutting edge 694 for collecting a biopsy sample from a patient, for example.

The reader will appreciate that the robotic system 682 can be used in combination with various dome-shaped and/or cylindrical magnetic housings further described herein. Moreover, the robotic system 682 and robotic tool 692 in FIG. 8 are exemplary. Alternative robotic systems can be utilized in connection with the various MRI systems disclosed herein. Moreover, handheld surgical instruments and/or additional imaging devices (e.g. an endoscope) and/or systems can also be utilized in connection with the various MRI systems disclosed herein.

In various aspects of the present disclosure, the MRI systems described herein can comprise low field strength MRI (LF-MRI) systems. In such instances, the main magnetic field B₀ generated by the permanent magnet assembly can be between 0.1 T and 1.0 T, for example. In other instances, the MRI systems described herein can comprise ultra-low field strength MRI (ULF-MRI) systems. In such instances, the main magnetic field B₀ generated by the permanent magnet assembly can be between 0.03 T and 0.1 T, for example.

Higher magnetic fields, such as magnetic fields above 1.0 T, for example, can preclude the use of certain electrical and mechanical components in the vicinity of the MRI scanner. For example, the existence of surgical instruments and/or surgical robot components comprising metal, specially ferrous metals, can be dangerous in the vicinity of higher magnetic fields because such tools can be drawn toward the source of magnetization. Moreover, higher magnetic fields often require specifically-designed rooms with additional precautions and shielding to limit magnetic interference. Despite the limitations on high field strength MRI (HF-MRI) systems, low field and ultra-low field MRI systems present various challenges to the acquisition of high quality images with sufficient resolution and quality for achieving the desired imaging objectives.

LF-MRI and ULF-MRI systems generally define an overall magnetic field homogeneity that is relatively poor in comparison to higher field MRI systems. For example, a dome-shaped housing for an array of magnets, as further described herein, can comprise a Halbach array of permanent magnets, which generate a magnetic field B₀ having a homogeneity between 1,000 ppm and 10,000 ppm in the region of interest in various aspects of the present disclosure.

In various instances, the intrinsic MRI signal is proportional to field strength. Consequently, the signal-to-noise (SNR) associated with a LF- and ULF-MRI system can theoretically be more than twenty times lower than the SNR for a high-field MRI system. For example, for a 70 mT MRI system, the SNR can be approximately 5% the SNR for a 1.5 T MRI system.

LF-MRIs exhibit a high degree of spatial distortion when acquired using a heterogeneous main magnetic field. The main magnetic field of the LF-MRI can be between 0.05 T and 1.0 T. In some aspects, the heterogeneous main magnetic field is generated from an array of small permanent magnets, e.g. Hallbach arrays. The main field homogeneity can range from parts per thousand (ppt) to as much as a percent variation across the usable imaging volume. These variations can cause similarly large phase and/or frequency shifts during the imaging pulse sequence, leading to distortions due to spatial errors (translations) as much as several centimeters, as well as undesirable rotations and/or shearing, for example. Clinical HF-MRI, on the other hand, is typically performed in a superconducting main magnetic field between 1T and 3T, for example, where the imaging volume homogeneity is better than 5 parts per million (ppm) over a 30-cm diameter spherical volume. Clinical HF-MRI scans generally exhibit very low spatial distortions that are typically at the pixel or sub-pixel level, which corresponds to mm-level errors.

In various aspects, machine learning approaches can be applied to LF-MRI for distortion correction to generate higher quality images. LF-MRI systems provide many benefits, such as allowing for interventional and image-guided neurological MRI procedures, e.g. brain biopsy and tumor resection/ablation. For example, intraoperative MRIs, i.e. MR imaging obtained during a surgical operation, can be used to provide guidance to a surgical tool appearing in the image. In various instances, interventional and image-guided neurological MRI procedures generally rely on one or more prior HF-MRI or high-resolution computed tomography (CT) image scans for procedure planning. Such prior HF-MRI or high-resolution CT images generally have very low spatial distortion and provide the possibility of pairing the HF-MRI or high-resolution CT image of a patient together with a more distorted LF-MRI scan that can be acquired at the time of a surgical procedure. The two images can used to train a machine learning model (e.g. a generative adversarial network (GAN)) to synthesize a distortion-corrected image. In various instances, multiple HF-MRIs and/or high-resolution CT images can be paired with the LF-MRI. During a surgical procedure, a LF-MRI can be input into the machine learning model to synthesize a distortion-corrected image.

The machine learning approach can be trained on pairs of a high-resolution image (e.g. HF-MRI and/or high-resolution CT image) and a LF-MRI from one or more subjects until the distortion-corrected images output from the machine learning model are considered to be suitable for the requirements of the interventional or image-guided procedures. In various instances, expert neuroradiologists, neurologists, and/or neurosurgeons can determine when the distortion-corrected images are suitable. The high-resolution image and LF-MRI are paired because the images are of the same object of interest from the same subject (e.g. the same organ in the same patient). In various instances, the paired images can be obtained within a predefined timeframe, such as within three days, 24 hours, or 8 hours, for example. In some aspects, the target image used with the LF-MRI in the machine learning model is a HF-MRI. In an alternative aspect, the target image might be a high-resolution CT image of the object of interest where key features are present, e.g. brain margins, ventricles, and skull position. In yet another alternative aspect, the target image is a combination of both the HF-MRI and high-resolution CT image (e.g. using both a HF-MRI and a high-resolution CT image for the target image).

In some aspects, it is beneficial to modify the high-resolution image to more closely resemble the generated output image (that is based on the LF-MRI) for use with the machine learning model. In one aspect, one or more intermediate image targets might be used with the machine learning model, e.g. the intermediate target might be a modified HF-MRI or modified high-resolution CT image where the HF-MRI or high-resolution CT image is modified to have a reduced spatial resolution (i.e. smoothed) in two or three dimensions such that the modified HF-MRI or modified high-resolution CT image more closely resembles the spatial resolution of the LF-MRI and the generated, distortion-corrected output image. The appearance of the paired target (e.g. HF-MRI or high-resolution CT image) and the LF-MRI might initially be made as similar as possible. For example, the image contrast for the paired images can be matched to be FLAIR, T2-weighted, T1-weighted, and so on.

In some aspects, training the machine learning model is a multi-stage process. For example, the machine learning model might initially be trained with LF-MRI and an intermediate target image, e.g., a modified HF-MRI or modified high-resolution CT image. As the performance improves, the process could be changed so that the target is swapped from a modified HF-MRI or modified high-resolution CT image (lower resolution image) as an intermediate to a high resolution HF-MRI. Stated another way, as the machine learning model is trained and improved over time, a larger difference, or at least larger potential difference, between the target, which is considered to be “distortion-free”, and the generated output image can be accommodated by the model.

Upon outputting the improved image from the machine learning model based on the LF-MRI image of the subject, the improved image can be further refined to produce the final distortion-corrected LF-MRI for a patient. For example, a refining step can include up-sampling the output synthetic, distortion-corrected image to match a template image resolution for the purposes of registration, for example. Additionally or alternatively, a refining step can include the application of an intensity correction to produce a more uniform image contrast. In some aspects, one or more modified or additional HF-MRI or high-resolution CT scans may be acquired along with LF-MRIs before the surgical procedure and can be used to refine the machine learning model, in order to maximize the accuracy and fidelity of the distortion-corrected LF-MRIs.

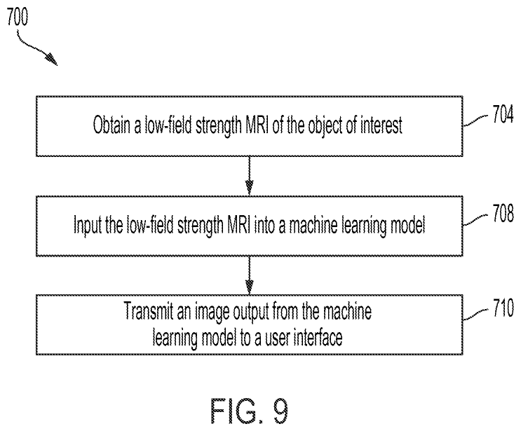

FIG. 9 depicts a method 700 for removing distortions from a LF-MRI using a pre-trained machine learning model, where the machine learning model is trained on at least one LF-MRI and a paired high-resolution image (e.g. HF-MRI or high-resolution CT image). The LF-MRI may be collected during a surgical procedure with a LF-MRI or ULF-MRI system, such as the MRI scanning system 100 including any of the various dome-shaped housings and magnetic arrays and/or the MRI system 500, for example. In various aspects, the method 700 and/or portions thereof can be implemented by a computing device, e.g. one or more control circuits of the computer 542 (FIG. 6).

The method 700 includes the control circuit obtaining 704, during the surgical procedure, i.e. intraoperatively, a LF-MRI of the object of interest. The LF-MRI was generated with a low-field strength magnetic field. In various aspects, the LF-MRI is generated by MRI scanning system 100 including any of the various dome-shaped housings and magnetic arrays or the MRI system 500, for example. In at least one aspect, the LF-MRI includes at least one distortion. The distortion may be caused by inhomogeneity in the low-strength magnet field, nonlinearity in gradient coil magnetic fields, and/or eddy currents associated with the switching of the gradient coils. In at least one aspect, the distortion is a distortion of the anatomical structure, or object of interest, depicted in the LF-MRI. Some examples of distortions are spatial errors (translations), which can be as large as several centimeters, for example, undesirable rotations, shearing, and coalescence of signals into the same pixel or voxel of the image.

The method 700 further includes the control circuit inputting 708, during the surgical procedure, the LF-MRI of the object of interest into a machine learning model. In at least one aspect, the machine learning model includes a generator model of a GAN. In various aspects, the machine learning model runs on the control circuit and is unsupervised.

The machine learning model outputs a distortion-corrected image of the object of interest based on the LF-MRI in real-time, e.g. during the surgical procedure. In at least one aspect, the machine learning model minimizes any distortions in the LF-MRI to create the outputted image. The method 700 further includes the control circuit transmitting 710 the distortion-corrected image, during the surgical procedure, to a user interface, e.g. a display of the computer 542, or any other device that allows a user to view the image.

As one example, a GAN can be used to train a generator model for intra-operatively generating an image based on a LF-MRI of the object of interest. For example, a GAN can be used to train a generator model used in the method 700 for generating a distortion-corrected image. A GAN is comprised of two models that work in competition with each other: a generator model and a discriminator model. The generator model comprises a first neural network that can be used to generate an image. The discriminator model comprises a second neural network that evaluates images to determine if a given image is “real” or “synthetic” (i.e., “fake”). The generator model creates synthetic, or fake, LF-MRI images with corrected distortions from real LF-MRI, while the discriminator decides whether the input image is “real” or “synethic”, i.e. generated by the generator. Stated another way, the discriminator model determines if the generated image by the generator model is of the same set or type of image as the real image or not. These two models work together such that the goal of the generator model is to have the discriminator model classify the generated images as “real” and the goal of the discriminator model is to correctly classify the generated images as “synthetic”.

The two models are updated to improve their goal. For example, the generator neural network can be penalized when the discriminator neural network determines that the generated image is fake. The generator can be rewarded when the discriminator decides the sythetic image is real. Similarly, the discriminator neural network can be penalized if the discriminator neural network determines that the generated image is real or if it decides a real image is fake. Moreover, the discriminator neural network can be rewarded when it correctly determines that the generated image is synthetic and a real image is real. In various instances, the process utilized by the GAN can improve the overall quality of the images produced by the generator model as the generator model and the discriminator model improve, i.e., as the generator model generates better images and as the discriminator model finds it harder to distinguish synthetic images from real images.

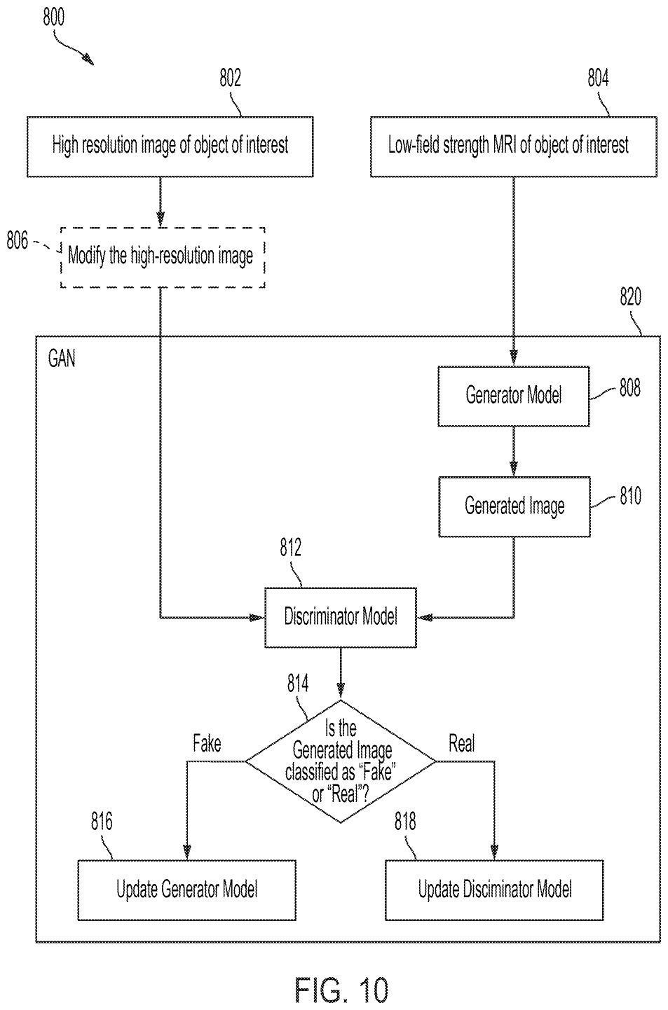

A GAN can be trained with pairs of high-resolution images and LF-MRIs from a plurality of test subjects, where each pair is a high-resolution image and a LF-MRI for the same test subject. Some of the pairs can be between a high-resolution CT image and a LF-MRI from the same subject and some of the pairs can be between a HF-MRI and a LF-MRI from the same subject. For example, the LF-MRI and high-resolution image can be input into the GAN, where the generator model receives the LF-MRI and the discriminator model receives the high-resolution image and a distortion-corrected image output from the generator model. The generator model can generate an image based off of the LF-MRI. The high-resolution image (e.g. the HF-MRI or high-resolution CT image) and the image from the generator model can be input into the discriminator model. The generator model and the discriminator model can be trained on multiple LF-MRI and high-resolution image pairs until the images generated by the generator model (i.e. distortion-corrected LF-MRIs) are considered to be suitable for the requirements of interventional and/or image-guided procedures. This process is described in more detail in regard to FIG. 10.

FIG. 10 depicts a method 800 for training a GAN to remove distortions from LF-MRI using paired HF-MRI. In various aspects, the LF-MRI is collected prior to a surgical procedure, i.e. pre-operatively, with a LF-MRI or ULF-MRI system, such as the MRI scanning system 100 including any of the various dome-shaped housings and magnetic arrays and/or the MRI system 500, for example. In various aspects, the method 800 and/or portions thereof can be implemented by a computing device, e.g. one or more control circuits of the computer 542 (FIG. 6).

The method 800 includes the control circuit obtaining 802 a high-resolution image of a first object of interest, e.g. a brain of a patient. In at least one aspect, the high resolution image is a HF-MRI and the HF-MRI is generated with a high-field strength magnetic field, as described previously herein. In an alternative aspect, the high-resolution image is a high-resolution CT image. In various aspects, the high-resolution image is collected prior to a surgical procedure, i.e. pre-operatively. The high-resolution image can be either a HF-MRI or a high resolution CT image and the following processes of method 800 remain the same.

The method 800 further includes the control circuit obtaining 804, preoperatively, a LF-MRI of the object of interest. The LF-MRI was generated with a low-field strength magnetic field. The high-resolution image and the low-field strength image are paired images of the same subject acquired within a relatively short period of time. In various aspects, the LF-MRI is generated by MRI scanning system 100 including any of the various dome-shaped housings and magnetic arrays, or the MRI system 500. In at least one aspect, the LF-MRI includes at least one distortion.

In some aspects, the method 800 further includes the control circuit modifying 806 the high-resolution image. The modification can convert the high-resolution image (e.g. the HF-MRI or the high-resolution CT image) into an image that is a closer match to the LF-MRI. In at least one aspect, a spatial resolution of the high-resolution image is greater than a spatial resolution of the LF-MRI. In one aspect, the spatial resolution of the high-resolution image is adjusted to match the spatial resolution of the LF-MRI. For example, high-resolution image can be smoothed to adjust the spatial resolution to match the spatial resolution of the LF-MRI. The modified high-resolution image can be used as an intermediate target during training of the machine learning model. The modified high-resolution image can provide a benefit of more closely matching the LF-MRI than the original high-resolution image. In some alternative aspects, modifying 806 the high-resolution image may not be desired and/or necessary, and, as such, the control circuit may not modify the high-resolution image prior to inputting the image into the machine learning model.

In at least one aspect, the method 800 includes prior training of the GAN, where the training has been performed using a plurality of paired high-resolution images and LF-MRIs obtained from healthy volunteers and patients in earlier imaging sessions. During training, the discriminator model is trained directly on real and generated images and is responsible for classifying images as “real” or “fake”, while the generator model is trained using feedback from the discriminator model, as further described herein. The training period seeks the minimization of appropriate loss functions for the generator model and the discriminator model together when applied to the training dataset. In one aspect, the discriminator model seeks to maximize the probability that it correctly assigns a “real” label to real HF images and a “fake” label to synthetic LF images it receives from the generator model. At the same time, the generator model seeks to maximize the probability that its synthetic LF-MRI images are classified as “real” by the discriminator model.

The HF-MRI and the LF-MRI are input into the GAN 820. The method 800 further includes the control circuit inputting the LF-MRI and high-resolution image (or modified high-resolution image) into the GAN 820. The method 800 further includes the control circuit inputting 808 the LF-MRI into the generator model of the GAN. The method 800 further includes the generator model outputting 810, by the control circuit, a generated image based on the LF-MRI. Distortions in the LF-MRI were minimized to produce the generated image. The method 800 further includes the control circuit inputting 812 the generated image and the high-resolution image (or modified high-resolution image) into the discriminator model. The method 800 further includes the control circuit determining 814 based on an output from the discriminator model if the generated image is classified as “real” or “fake.” The discriminator model seeks to maximize the correct probability assigned to “real” and “fake” images, while the generator model either seeks to minimize the probability of its images are predicted as “fake” or it seeks to maximize the probability of images being predicted as “real”.

If the discriminator model determines that the generated image is “fake”, then the method 800 proceeds to the control circuit updating 816 the generator model. The control circuit can update the generator model by adjusting the weights and/or biases of the neural network of the generator model. In at least one aspect, the adjustments are made with an optimization approach, where the generator model either seeks to minimize the probability of its images being predicted as “fake” or it seeks to maximize the probability of images being predicted as “real”. The update to the generator model is to improve the generated images produced by the generator model. For example, as the generator model is updated the generated images will begin to increasingly look more like the high-resolution image.

If the discriminator model determines that the generated image is “real”, then the method 800 proceeds to the control circuit updating 818 the discriminator model. The control circuit can update the discriminator model by adjusting the weights and/or biases of the neural network of the discriminator model. In at least one aspect, the adjustments are made with an optimization approach, where the discriminator model seeks to maximize the correct probability assigned to “real” and “fake” images. For example, the update to the discriminator model is to improve the classification of the generated images as “fake” and the high-resolution images as “real”. Both the discriminator model and the generator model can improve during training so that one model is not improved to be not substantially better than the other model.

During the training of the GAN, simultaneous improvements are made to both the generator and discriminator models that are in competition with each other. Once an update is made to either the generator model or the discriminator model, then the method 800 can be repeated to keep updating the GAN. This updating process leads the generator model to produce better quality images based on the LF-MRI image. For example, the generator model is trained to produce higher quality images as the generator model is optimized, and the discriminator is trained to improve at detecting fake images as the discriminator model is optimized. As the quality of a generated image improves, more distortions can be removed from the generated image and the generated image can look more like the high-resolution image.

The GAN is trained to a desired performance level. For example, the GAN can be trained until the distortion-corrected images output from the generator model are considered to be suitable for the requirements of the interventional or image-guided procedures. In various instances, expert neuro-radiologists, neurologists, and/or neurosurgeons can determine if the distortion-corrected images are suitable. The method 800 further includes the control circuit transmitting a notification to a user interface based on the generative adversarial network reaching the desired performance level. For example, once the control circuit determines that the generative adversarial network has reached the desired performance level, then an expert can review a distortion-corrected image to determine if the distortion-corrected images are suitable for interventional or image-guided procedures.

In at least one aspect, different pairs of high-resolution images (e.g HF-MRIs or high-resolution CT images) and LF-MRIs can be used during the training of the GAN. For example, a plurality of paired high-resolution images and LF-MRIs are used during the training. In various aspects, a new pair of high-resolution images and LF-MRIs can be used for each iteration of the method 800 or the same pair of high-resolution images and LF-MRIs can be used multiple times before changing to a new pair of high-resolution images and LF-MRIs. Paired high-resolution images and LF-MRIs are images of the same object of interest from the same patient. If the high-resolution image is a HF-MRI, then one MRI is collected with a high field strength magnetic field and one MRI is collected with a low-field strength magnetic field. Using more than one pair of high-resolution images and LF-MRIs can provide the GAN with different images and subjects to train and can improve the quality of the machine learning tool in various instances. As an example, the GAN can be trained with several hundred matched pairs of LF-MRI and HF-MRI from a variety of patient types. The patients may include healthy people in addition to people with strokes, people who have had tumor resections, people who have had craniotomies, and/or people who have significant midline shift due to hydrocephalus ex vacuo, for example. In certain instances, training the model on a variety of anatomical structures, including highly abnormal ones (e.g. cases where the skull may be missing) may be advantageous.

In at least one aspect, the generator model is trained without updating the discriminator model and then the discriminator model is trained without updating the generator model, or vice versa. This approach can provide the benefit of training one of the models without the other model changing, which can allow one of the models to improve in regard to the other model. This process can also simplify the optimization of improving the model being trained since the model is being trained against something that is not changing. This approach can also be beneficial if one of the models is not working as well as the other model. For a GAN to operate properly, the two models need to be at a similar level of quality of results. For example, upon the discriminator model only detecting a “fake” image for approximately half of the total images, a tool can be considered to be sufficient quality and the generator model and the discriminator model thereof can be considered to be sufficiently similar in quality.

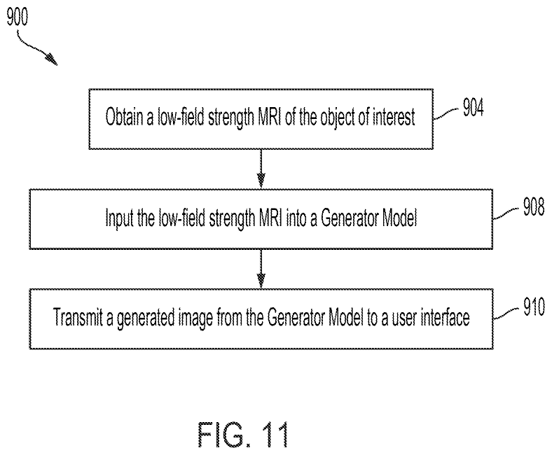

FIG. 11 depicts a method 900 for using a generator model trained by a generative adversarial network (e.g. as discussed in regard to method 800 of FIG. 10) to remove distortions from a LF-MRI during a surgical procedure. Similar to the method 700, the LF-MRI may be collected during a surgical procedure with a LF-MRI or ULF-MRI system, such as the MRI scanning system 100 including any of the various dome-shaped housings and magnetic arrays or the MRI system 500, for example. In various aspects, the method 900 and/or portions thereof can be implemented by a computing device, such as one or more control circuits of the computer 542 (FIG. 6), for example.

The method 900 includes the control circuit obtaining 904, during the surgical procedure, i.e. intraoperatively, a LF-MRI of the object of interest. In various aspects, the LF-MRI is generated by MRI scanning system 100 including any of the various dome-shaped housings and magnetic arrays or the MRI system 500, for example. In at least one aspect, the LF-MRI includes at least one distortion. The method 900 further includes the control circuit inputting 908 the LF-MRI into a generator model of the GAN and the generator model outputting a generated image based on the LF-MRI, wherein distortions in the LF-MRI are minimized to produce the generated image. Thereafter, the method 900 further includes the control circuit transmitting 910 the generated image to a user interface, such as a display of the computer 542, for example, or any other device that allows a user to view the image.

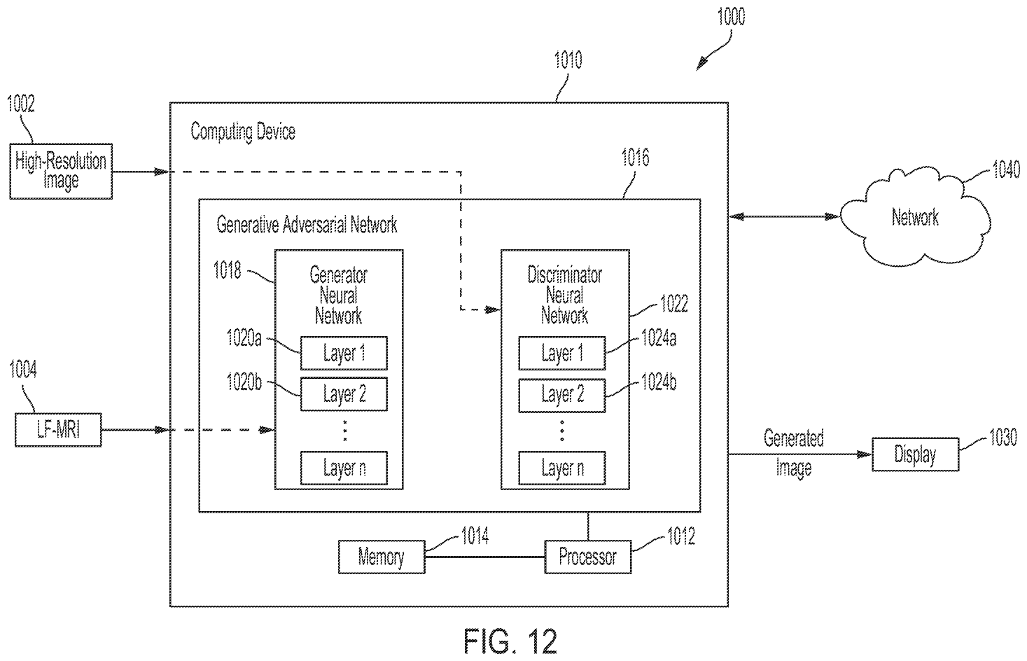

FIG. 12 depicts a system 1000 for using a machine learning model to remove distortions from a LF-MRI 1004 during a surgical procedure. The system 1000 includes a computing device 1010 having a memory 1014, a processor 1012, and a machine learning model 1016, which is a generative adversarial network. The computing device 1010 may communicate with one or more other computing devices over a network 1040. The computing device 1010 may be implemented as a server, a desktop computer, a laptop computer and/or a mobile device, such as a tablet device or mobile phone device, for example. In various instances, the computing device 1010 may be representative of multiple computing devices in communication with one another, such as multiple servers in communication with each another.

The processor 1012 may represent two or more processors on the computing device 1010 executing in parallel and utilizing corresponding instructions stored using the memory 1014. The memory 1014 represents a non-transitory computer-readable storage medium. The memory 1014 may represent one or more different types of memory utilized by the computing device 1010, for example. In addition to storing instructions, which allow the processor 1012 to implement the machine learning model 1016 and computer-readable instructions stored in the memory 1014, the memory 1014 may be used to store data, e.g. imaging data, pre-trained models such as machine learning models, algorithms, and/or subroutines thereof, for example.

A user may input a LF-MRI 1004 and a paired high-resolution image 1002 (e.g. a HF-MRI or a high-resolution CT image) into a machine learning model 1016 of the computing device 1010 during training of the machine learning model. In other aspects, the machine learning model 1016 may be running on the computing device 1010 as a component of a cloud network where a user inputs a LF-MRI 1004 and a paired high-resolution image 1002 during training from another computing device over a network, such as the network 1040. After training of the machine learning model 1016 to a desired performance level, the machine learning model 1016 can generate images with distortions removed based on a LF-MRI without a high-resolution image. The LF-MRI can be collected with an MRI scanning system. For example, the MRI scanning system can be MRI scanning system 100 including any of the various dome-shaped housings and magnetic arrays or the MRI system 500. The machine learning model 1016 enables distortions to be removed in real-time based on the paired LF-MRI 1004 and the high-resolution image 1002.

The machine learning model 1016 illustrated in FIG. 12 is a generative adversarial network, which includes a generator model and a discriminator model. The generator model is a generator neural network 1018 that includes a plurality of layers 1020a, 1020b, etc. The discriminator model is a discriminator neural network 1022 that includes a plurality of layers 1024a, 1024b, etc. The generator neural network 1018 and the discriminator neural network 1022 could have any number of layers. While FIG. 12, shows the generator neural network 1018 and the discriminator neural network 1022 having at least 3 layers, the neural networks could have only 1 or 2 layers. Additionally, while the generator neural network 1018 and the discriminator neural network 1022 can have the same number of layers, they do not need to have the same number of layers.

As discussed in regard to FIGS. 10, during training of the machine learning model, or GAN, 1016, the LF-MRI 1004 and high-resolution image 1002 are input into the GAN 1016. The LF-MRI 1004 is input into the generator neural network 1018 and the high-resolution image 1002 is input into the discriminator neural network 1022. The discriminator neural network 1022 receives a generated image from the generator neural network 1018 along with the high-resolution image 1002. The discriminator neural network 1022 and the generator neural network 1018 are simultaneously trained as discussed in regard to FIG. 10. Similar to the method 800, the high-resolution image 1002 can be a HF-MRI or a high-resolution CT image. The generator neural network 1018 and discriminator neural network 1022 are trained pre-operatively. The trained generator neural network 1018 is used intra-operatively as discussed in regard to method 900 of FIG. 11.

In various aspects, upon receiving a paired high-resolution image 1002 and LF-MRI 1004, the processor 1012 of the computing device 1010 can implement training of the machine learning model 1016 to train the generative adversarial network based on the paired high-resolution image 1002 and LF-MRI 1004. For example, the machine-readable instructions stored in the memory 1014 can run the machine learning model 1016 via the processor 1012 to train the generator neural network 1018 and the discriminator neural network 1022 in accordance with the method 800 (FIG. 10), for example. In at least one aspect, the LF-MRI 1004 and high-resolution image 1002 are images of the same object of interest from the same patient.

During a surgical procedure, a LF-MRI 1004 can be collected with an MRI scanning system. For example, the MRI scanning system can be MRI scanning system 100 including any of the various dome-shaped housings and magnetic arrays or the MRI system 500. The LF-MRI 1004 is input into the generator neural network 1018. The generator neural network 1018 produces a generated image that has any distortions of the LF-MRI minimized. The generated image is output to a display 1030 based on an output of the generator neural network 1018.

In various aspects, upon receiving a LF-MRI 1004, the processor 1012 of the computing device 1010 can implement the machine learning model 1016 to apply the generator neural network 1018 to the LF-MRI 1004. For example, the machine-readable instructions stored in the memory 1014 can run the generator neural network 1018 via the processor 1012 to generate an image with minimal distortions, in real-time, of an object of interest based on a LF-MRI of an object of interest from a patient in accordance with the method 700 (FIG. 9) and method 900 (FIG. 11), for example.

Though various aspects disclosed herein are directed to brain imaging and/or neurological interventions, the reader will appreciate that the various systems and methods disclosed herein can be used to image other portions of a patient's anatomy and/or different structures in various instances.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a control circuit computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material. In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A system, comprising:
a database storing a preoperative high-resolution image of an object of interest; and
a control circuit comprising a processor and a memory, wherein the memory stores instructions executable by the processor to:
obtain, intraoperatively, a low-field strength magnetic resonance image (MRI) of the object of interest;
input, intraoperatively, the low-field strength MRI of the object of interest into a generator model of a pre-trained generative adversarial network, wherein the generator model is pre-trained with low-field strength MRIs and paired high-resolution images to correct image distortions;
output, intraoperatively, a distortion-corrected image of the object of interest from the generator model based on the low-field strength MRI; and
transmit, intraoperatively, the distortion-corrected image of the object of interest to a user interface.

2. The system of claim 1, wherein the paired high-resolution images are images selected from a group consisting of a high-field strength MRI and a high-resolution computed tomography image.

3. The system of claim 1, wherein the generative adversarial network is trained to a desired performance level using at least one set of training images, wherein each set of training images comprises a high-resolution training image of a training object of interest and low-field strength MRI training image of the training object of interest, and wherein the system further comprises a training control circuit to:
obtain, preoperatively, a first set of training images, wherein the first set of training images comprises a first low-field strength MRI training image and a first high-resolution training image of a first training object of interest;
input, preoperatively, the first low-field strength MRI training image into the generator model of the generative adversarial network to generate a first distortion-corrected training image;
input, preoperatively, the first distortion-corrected training image and the first high-resolution training image into a discriminator model of the generative adversarial network to evaluate the first training image; and
update, preoperatively, one of the generator model and the discriminator model based on the evaluation of the first distortion-corrected training image by the discriminator model.

4. The system of claim 3, wherein the discriminator model comprises a discriminator neural network and the generator model comprises a generator neural network, and wherein the training control circuit is further to adjust at least one weight of at least one layer of the discriminator neural network based on the discriminator model classifying the first distortion-corrected training image from the generator model as “real”.

5. The system of claim 3, wherein the discriminator model comprises a discriminator neural network and the generator model comprises a generator neural network, and wherein the training control circuit is further to adjust at least one weight of at least one layer of the generator neural network of the generator model based on the discriminator model classifying the first distortion-corrected training image as “fake”.

6. The system of claim 1, wherein the low-field strength MRI comprises a distortion of an anatomical structure depicted in the low-field strength MRI.

7. The system of claim 1, wherein the paired high-resolution images comprise a first resolution, wherein the low-field strength MRIs comprises a second resolution, wherein the second resolution is less than the first resolution, wherein the memory stores further instructions executable by the processor to adjust the first resolution of the paired high-resolution images based on the second resolution of the low-field strength MRIs prior to training the generative adversarial network.

8. The system of claim 1, wherein adjusting the first resolution based on the second resolution comprises smoothing each paired high-resolution image.

9. The system of claim 1, wherein the memory stores further instructions executable by the processor to generate, intraoperatively, a low-field strength MRI with a low-field strength magnetic field.

10. The system of claim 9, further comprising:
a dome-shaped housing that is configured to house an array of magnets, wherein the array of magnets are arranged to generate the low-field strength magnetic field toward the object of interest within a field of view, wherein the low-field strength magnetic field comprises a magnetic field strength less than or equal to 1 T; and
a radio frequency coil assembly configured to selectively excite magnetization in the object of interest in the field of view.

11. The system of claim 1, wherein the memory stores further instructions executable by the processor to transmit, intraoperatively, the distortion-corrected image of the object of interest to the user interface in real time.

12. The system of claim 1, wherein the object of interest comprises an anatomical structure of a particular patient.

13. A training system for a generative adversarial network, the training system comprising:
a training processor; and
a training memory storing a plurality of sets of training images, wherein each set of training images comprises a high-resolution training image of a training object of interest and a paired low-field strength MRI training image of the training object of interest, and wherein the memory stores instructions executable by the processor to:
obtain a first set of training images, wherein the first set of training images comprises a first low-field strength MRI training image and a first high-resolution training image of a first training object of interest;
input the first low-field strength MRI training image into a generator model of a generative adversarial network to generate a first distortion-corrected training image;
input the first distortion-corrected training image and the first high-resolution training image into a discriminator model of the generative adversarial network;
evaluate, by the discriminator model, the first distortion-corrected training image to identify the first distortion-corrected training image as one of “real” or “fake”; and
update, preoperatively, the generative adversarial network based on the evaluation of the first distortion-corrected training image by the discriminator model, wherein updating the generative adversarial network comprises:
if the discriminator model classified the first distortion-corrected training image from the generator model as “real”, updating the discriminator model; and
if the discriminator model classified the first distortion-corrected training image from the generator model as “fake”, updating the generator model.

14. The training system of claim 13, wherein the discriminator model comprises a discriminator neural network and the generator model comprises a generator neural network, and wherein the training memory stores instructions executable by the training processor to:
adjust at least one weight of at least one layer of the discriminator neural network of the discriminator model classified the first distortion-corrected training image from the generator model as “real”; and
adjust at least one weight of at least one layer of the generator neural network of the generator model based on the discriminator model classifying the first distortion-corrected training image as “fake”.

15. The training system of claim 13, further comprising training the generator model to a desired performance level by:
obtaining at least one other set of training images of a different subject; and
further training the generative adversarial network with the at least one other set of training images.

16. A method, comprising:
training, preoperatively, a generative adversarial network to a desired performance level, wherein training the generative adversarial network comprises:
inputting a first set of training images into the generative adversarial network, wherein the first set of training images comprises a first low-field strength MRI training image and a first high-resolution training image of a first training object of interest, and wherein the generative adversarial network comprises a generator model and a discriminator model;
generating, by the generator model, a first distortion-corrected training image based on the first low-field strength MRI training image;
receiving, by the discriminator model, the first distortion-corrected training image and the first high-resolution training image;
classifying, by the discriminator model, the first distortion-corrected training image as “real” or “fake”; and
updating the generative adversarial network based on the classification, wherein updating the generative adversarial network comprises:
updating the discriminator model if the first distortion-corrected training image was classified as “real”; and
updating the generator model if the first distortion-corrected training image was classified a “fake”; the method further comprising:
transmitting a notification to a user interface based on the generative adversarial network reaching the desired performance level.

17. The method of claim 16, further comprising obtaining, preoperatively, a high-field strength MRI of the first training object of interest.

18. The method of claim 16, further comprising obtaining, preoperatively, a high-resolution computed tomography image of the first training object of interest.

19. The method of claim 16, further comprising, after training the generative adversarial network to the desired performance level, generating a distortion-corrected image with minimized distortions, wherein generating the distortion-corrected image comprises:
obtaining, intraoperatively, a low-field strength MRI of an object of interest with a low-field strength magnetic resonance imaging system, wherein the low-field strength MRI comprises a dome-shaped housing and an array of magnets arranged about the dome-shaped housing;
inputting, intraoperatively, the low-field strength MRI of the object of interest into the generator model, wherein the generator model is to generate the distortion-corrected image; and
transmitting, intraoperatively, the distortion-corrected image to a user interface, wherein generation and transmission of the distortion-corrected image occurs in real-time.

20. The method of claim 19, further comprising:
projecting a low-field strength magnetic field from the array of magnets toward the object of interest located within a field of view, wherein the low-field strength magnetic field comprises a magnetic field strength less than or equal to 1 T;
transmitting a radio frequency pulse sequence to a radio frequency coil assembly configured to selectively excite magnetization in the object of interest within the field of view; and
receiving and recording an output signal from the radio frequency coil assembly.
